(19) United States
(12) Patent Application Publication
(10) Pub. No.: US 2025/0152519 A1
(43) Pub. Date: May 15, 2025

(54) DRESSINGS AND MEDICATED PADS FOR REMOVING SKIN DISORDERS

(71) Applicant: DelStar Technologies, Inc., Alpharetta, GA (US)

(72) Inventor: Nick SHAFFER, Alpharetta, GA (US)

(21) Appl. No.: 18/938,595

(22) Filed: Nov. 6, 2024

Related U.S. Application Data
(60) Provisional application No. 63/598,177, filed on Nov. 13, 2023.

(51) Int. Cl. A61K 9/70; A61K 31/60; A61K 47/32

(52) U.S. Cl. CPC A61K 9/7084; A61K 31/60; A61K 47/32

(57) ABSTRACT

The present disclosure provides medicated pads, bandages and/or dressings for removing skin disorders, diseases and conditions, such as corns, calluses, blisters, warts, acne, and the like. A medical dressing comprises a first layer having an adhesive surface and a second layer disposed adjacent to the adhesive surface of the first layer and comprising an active agent. The dressing further includes a third layer comprising hydrogel spaced from and at least partially surrounding the second layer. The dressing, the hydrogel and the active agent may be applied to the skin in a single step, resulting in a faster and easier process for the user. The hydrogel layer moisturizes the skin around the affected area, while reducing the amount of pressure applied thereto, thereby providing more comfort for the user.

### CROSS-REFERENCE TO RELATED APPLICATIONS

This application claims the benefit of U.S. Provisional Application Ser. No. 63/598,177 filed Nov. 13, 2023, the complete disclosure of which is incorporated herein by reference in its entirety for all purposes.

### TECHNICAL FIELD

This description generally relates to dressings, bandages and/or pads for removing a portion of a user's skin and, more particularly to, medicated pads for removing corns, calluses, blisters, warts, acne or other skin disorders, diseases or conditions.

### BACKGROUND

Skin conditions, diseases and disorders may include any medical condition that affects the integumentary system or the skin, nails and related muscles and glands. Some of these skin conditions cause intense pain and discomfort. For example, corns are a painful type of hyperkeratosis, found principally over prominent toe joints and between toes. A callus may be a diffuse or circumscribed area of hyperkeratosis at a site of repeated pressure and friction. In cases where there is a forefoot imbalance, the plantar callus may be found where the metatarsal heads are most prominent. Plantar warts are simple papillomas caused by a virus. Plantar warts differ from calluses and are not necessarily found over bony prominences.

Medicated pads are known for placement over corns, calluses and warts. These pads typically contain an ingredient or active agent, such as salicylic acid in a rubber based vehicle. For example, such pads are Sold by Schering-Plough Healthcare Products, Inc. of Memphis, TN (currently Yellow Wood Partners of Boston, MA) under the house trademark “DR. SCHOLL'S”, and under the particular trademark “DURAGEL”.

These medicated pads typically comprise a dressing or bandage that includes hydrogel disposed on an internal or adhesive surface of the bandage. The hydrogel is designed to moisturize the user's skin around the affected area. The medicated pad containing the active agent or salicylic acid is typically formed as a separate component from the dressing. The user removes the medicated pad from its packaging and applies it onto the affected area of the skin. The outer bandage with the hydrogel is then placed over the medicated disc.

### SUMMARY

Medicated pads, bandages and/or dressings are provided for removing skin disorders, diseases and conditions, such as corns, calluses, blisters, warts, acne, and the like.

In one aspect, a dressing comprises a first layer having an adhesive surface and a second layer disposed adjacent to the adhesive surface of the first layer and comprising an active agent. The dressing further includes a third layer comprising hydrogel spaced from and at least partially surrounding the second layer. The hydrogel and the active agent are contained together in the same dressing and thus may be applied to the skin in a single step, resulting in a faster and easier process for the user.

The active agent may comprise any ingredient or medication that is suitable for removing a target area of the skin that includes a skin disorder or condition. Suitable medications include, but are not limited to, keratolytic agents, such as salicylic acid or a salt or ester thereof. In one embodiment, the active agent comprises salicylic acid in a percentage by weight of about 30% to about 50% or about 35% to about 45%, or between about 38% to about 42%.

In various embodiments, the second or medicated layer is spaced from the first or outer layer and the hydrogel layer is in contact with the outer layer. The hydrogel provides moisture to the user's skin in the area immediately surrounding the target region. The hydrogel layer at least partially surrounds the medicated layer, and is spaced from the medicated layer, to reduce the amount of pressure applied to the target region of the user's skin, thereby providing more comfort for the user.

In various embodiments, the hydrogel comprises water in a percentage of less than about 50% by weight of the third layer, or about 10% to about 50% by weight of the third layer, or about 10% to about 40% or about 10% to about 30%. In exemplary embodiments, the percentage of water is less than about 40%, or about 30% or less. The reduced water content in the hydrogel increases the stability and overall shelf-life of the dressing.

The hydrogel layer may further comprise an acrylic based monomer. Suitable monomers include, but are not limited to, acrylic based monomers, such as 2-Acrylamido-2-methylpropane sulfonic acid (AMPS), ethyl acrylate, ethylene-methyl acrylate, methyl methacrylate, 2-chlorotheyl vinyl ether, 2-hydroxethyl acrylate, hydroxyethyl methacrylate, butyl acrylate, trimethylolpropane triacrylate (TMPTA), amides, such as acrylamide or acrylic acid, dicarboxylic acids, such as itaconic acid or methylidene succinic acid and combinations thereof. In an exemplary embodiment, the monomer comprises AMPS.

The hydrogel layer may further include a filler material. Suitable fillers include, but are not limited to glycerol, hyaluronic acid, and the like.

In various embodiments, the medicated layer comprises a substantially circular disc. In one embodiment, the hydrogel layer comprises first and second components spaced from each other on opposite sides of the medicated disc. In one such embodiment, the first component has substantially the same shape as the second component. In another embodiment, the first component has a larger cross-sectional area than the second component.

The outer layer may comprise any suitable material for protecting the other layers, such as polyurethane, polyolefin, polyamide, polyester, polypropylene, polyethylene, ethylene vinyl acetate (EVA) and combinations thereof. In an exemplary embodiment, the outer layer comprises polyurethane.

In various embodiments, the dressing further includes an interlayer between the adhesive surface of the outer layer and the medicated disc. The interlayer protects the outer layer from being degraded by the active agent. Suitable materials for the interlayer include, but are not limited to, polyethylene terephthalate (PET), polyurethane, polyolefin, polyamide, polyester, polypropylene, polyethylene, ethylene vinyl acetate (EVA) and combinations thereof. In an exemplary embodiment, the interlayer comprises PET.

In various embodiments, the dressing further comprises a fourth layer or release liner removably coupled to the outer layer. Alternatively, the dressing may include a folded liner system. The medicated layer and the hydrogel layer contact the release liner such that they contact the user's skin after removal of the release liner. Suitable materials for the interlayer include, but are not limited to, polyethylene terephthalate (PET), polyurethane, polyolefin, polyamide, polyester, polypropylene, polyethylene, ethylene vinyl acetate (EVA) and combinations thereof. In an exemplary embodiment, the interlayer comprises PET.

In various embodiments, the dressing further comprises a release tab removably secured to the adhesive surface of the outer layer and overlying the release liner. The release tab may comprise any suitable material, such as paper, or a polymer film, and functions to allow the user to remove the release liner before applying the adhesive surface to the skin.

In another aspect, a medical device includes a medicated pad or dressing as described above. In various embodiments, the medical device comprises a corn remover, a callus remover, a blister remover and/or a wart remover.

In another aspect, a medicated pad or dressing for use in removing a corn from a user's skin is provided. The medicated pad comprises a first layer having an adhesive surface configured for contacting the user's skin, a second layer comprising an active agent configured to remove the corn and a third layer comprising hydrogel and spaced from and at least partially surrounding the second layer.

In various embodiments, the second or medicated layer comprises a substantially circular disc. The third layer comprises a first component and a second component spaced from the first component. The first and second components each have a semi-annular shape and are disposed on opposite sides of the circular disc

The active agent may comprise any ingredient or medication that is suitable for removing a target area of the skin that includes a skin disorder or condition. Suitable medications include, but are not limited to, keratolytic agents, such as salicylic acid or a salt or ester thereof. In one embodiment, the active agent comprises salicylic acid in a percentage by weight of about 30% to about 50% or about 35% to about 45%, or between about 38% to about 42%.

In various embodiments, the hydrogel comprises water in a percentage of less than about 50%, or about 10% to about 50%, or about 10% to about 40% or about 10% to about 30% by weight of the third layer. In exemplary embodiments, the percentage of water is less than about 40%, or about 30% or less.

In another aspect, a medicated pad for use in removing a callus from a user's skin is provided. The medicated pad comprises a first layer having an adhesive surface configured for contacting the user's skin, a second layer comprising an active agent configured to remove the callus and a third layer comprising hydrogel and spaced from and at least partially surrounding the second layer.

In various embodiments, the outer layer comprises a substantially ovoid shape and the second layer comprises a substantially circular disc. The third layer comprises a first component and a second component spaced from the first component. The first and second components are disposed on either side of the circular disc and the first component has a smaller cross-sectional area than the second component. In an exemplary embodiment, the second component comprises first and second end portions and a central portion. The central portion has a larger thickness than the first and second end portions.

The active agent may comprise any ingredient or medication that is suitable for removing a target area of the skin that includes a skin disorder or condition. Suitable medications include, but are not limited to, keratolytic agents, such as salicylic acid or a salt or ester thereof. In one embodiment, the active agent comprises salicylic acid in a percentage by weight of about 30% to about 50%, or about 35% to about 45%, or between about 38% to about 42%.

In various embodiments, the hydrogel comprises water in a percentage of less than about 50%, or about 10% to about 50%, or about 10% to about 40% or about 10% to about 30% by weight of the third layer. In exemplary embodiments, the percentage of water is less than about 40%, or about 30% or less.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a bottom view of a dressing or medicated pad;

FIG. 2 is an exploded view of the dressing of FIG. 1;

FIG. 3 is a bottom view of another dressing or medicated pad; and

FIG. 4 is an exploded view of the dressing of FIG. 3.

### DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

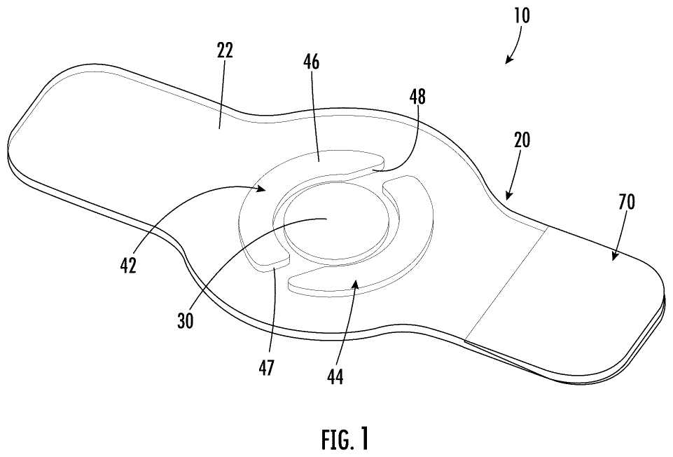

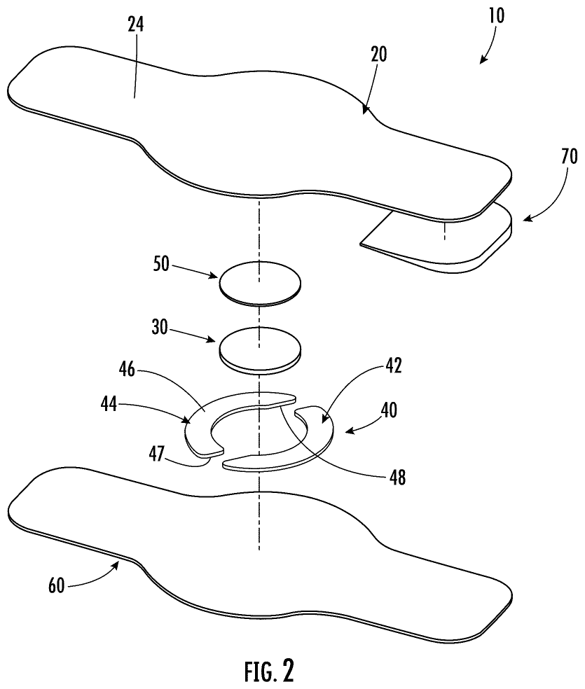

Referring now to FIGS. 1 and 2, a dressing or medicated pad 10 includes a first layer 20 having an outer surface 24 and an inner adhesive surface 22 configured to adhere to stick to a user's skin. Pad 10 is particularly useful for removing a corn from a user's skin, although it will be recognized that pad 10 may be used to treat and/or remove a variety of skin disorders, diseases or conditions, including blisters, calluses, acne, warts, psoriasis and the like.

First layer 20 may include any suitable material such as polyethylene, polyvinyl chloride (PVC), polyurethane (PU), polyester, ethylene vinyl acetate (EVA), polypropylene, polyethylene terephthalate (PET), polyurethane, polyolefin, polyamide and combinations thereof. In an exemplary embodiment, first layer 20 comprises a polyurethane material. First layer 20 may have a thickness of about 1 mil to about 5 mils, or about 3 mils.

First layer 20 may further include an adhesive material on surface 22, such as an acrylic pressure sensitive adhesive (PSA) or similar material. In certain embodiments, first layer 20 includes perforations or apertures to allow oxygen and water vapor to pass therethrough to keep the underlying skin dry while allowing it to breathe.

Pad 10 further includes a release layer 60 configured to removably attach to adhesive surface 22. Release layer 60 serves to protect adhesive surface 22 prior to application by the user. In addition, release layer 60 may serve to protect outer layer 20 from the active agent contained in second layer 30 (discussed below). Suitable materials for release layer 60 include, but are not limited to, polyethylene terephthalate (PET), polyurethane, polyolefin, polyamide, polyester, polypropylene, polyethylene, ethylene vinyl acetate (EVA), silicone, clay coated paper and combinations thereof. In an exemplary embodiment, release layer 60 comprises PET. Release layer 60 may have a thickness of about 1 mil to about 5 mils, or about 3 mils.

Pad 10 further comprises a release tab 70 removably secured to adhesive surface 22 of outer layer 20 and overlying release liner 60. Release tab 70 may comprise any suitable material, such as paper, or a polymer film, and functions to allow the user to remove release liner 60 before applying adhesive surface 22 to the skin.

Medicated pad 10 further includes a second layer 30 that includes an active agent suitable for removing a target region of a user's skin, such as a corn, callus, blister or the like. Suitable materials for the active agent include, but are not limited to, keratolytic agents, such as salicylic acid or esters or salts thereof, glacial acetic acid, phenoxy acetic acid, ascorbic acid, lactic acid, coal tar, pyrithione zinc, glycolic acid, phenol, podophyllum, zinc chloride, tannic acid, castor oil, retinoic acid (tretinoin), fluorouracil, calcium pantothenate, cantharidin, acitretin, olive oil and combinations thereof.

In one embodiment, the active agent comprises salicylic acid or a salt or ester thereof. Suitable salts include sodium, potassium, calcium or magnesium. Suitable esters include C3 or C4 esters, such as methyl salicylate, or salicylsalicylic acid. The salicylic acid may comprise about 6% to about 60% by weight of layer 30, or about 30% to about 50% or between about 38% to about 42%. Although second layer 30 is shown as a disc shape, second layer 30 may be any suitable shape. Second layer 30 preferably has a thickness of about 0.005 inches to about 0.1875 inches, or about 0.010 inches to about 0.035 inches.

Medicated pad 10 includes an interlayer 50 disposed between second layer 30 and outer layer 20. Interlayer 50 functions to protect outer layer 20 from the active agent within second layer 30 such that the active agent does not degrade outer layer 20. Interlayer 50 preferably has the same shape as second layer 30, although it will be recognized that interlayer 50 may include any suitable shape so long as it substantially inhibits or prevents contact between second layer 30 and outer layer 20. For example, interlayer 50 may have a larger cross-sectional area than second layer 30. Interlayer 50 may have a thickness of about 0.5 mils to about 5 mils, or about 1 mil. Suitable materials for interlayer 50 include, but are not limited to, polyethylene terephthalate (PET), polyurethane, polyolefin, polyamide, polyester, polypropylene, polyethylene, ethylene vinyl acetate (EVA) and combinations thereof. In an exemplary embodiment, interlayer 50 comprises PET.

Medicated pad 10 further includes a hydrogel layer 40 disposed between outer layer 20 and release liner 60. Hydrogel layer 40 provides moisture to the user's skin in the area immediately surrounding the target region. Hydrogel layer 40 at least partially surrounds second layer 30 and is spaced from second layer 30 such that it does not directly contact second layer 30. This configuration reduces the amount of pressure applied to the target region of the user's skin, which provides more comfort to the user. In addition, it reduces moisture transfer to the medicated pad.

In one embodiment, hydrogel layer 40 comprises first and second components 42, 44 each having an arcuate main body 46 extending from a first end 47 to a second end 48 and shaped to extend around at least a portion of the periphery of second layer 30. The first and second ends 47, 48 of each component 42, 44 are spaced from each other to form two separate hydrogel components that preferably do not contact each other. This configuration facilitates manufacturing of the medicated pad 10 and increases flexibility of the bandage. Each component 42, 44 of the hydrogel layer may have a thickness of about 0.010 inches to about 0.188 inches, or about 0.045 inches.

Other configurations of hydrogel layer 40 are contemplated. For example, hydrogel layer 40 may comprise a single substantially annular component that extends around the entire periphery of second layer 30. Alternatively, the single annular component may extend around only a portion of the second layer 30, i.e., from about 180 degrees to about 360 degrees. In other embodiments, hydrogel layer 40 may comprises more than two components. For example, hydrogel layer may comprise a plurality of smaller hydrogel components circumferentially spaced from each other around medicated layer 30. The individual hydrogel components may have any suitable shape, such as rectangular, square, circular, diamond, triangular and the like.

Hydrogel layer 40 preferably comprises one or more monomers and water. In certain embodiments, layer 40 may include a filler. The water content of hydrogel layer 40 is about 10% to about 50% by weight of layer 40, or about 10% to about 40% or about 10% to about 30%. In an exemplary embodiment, the weight percentage of water is 30% of layer 40. A water percentage of at least 50% or less, preferably 30% or less, inhibits degradation of the salicylic acid in second layer 30 and increases the stability or shelf-life of pad 10.

The monomer in hydrogel layer 40 preferably comprises at least one pendant polar group that functions to provide water solubility. Suitable monomers for user in hydrogel layer 40 include, but are not limited to, acrylic based monomers, such as 2-Acrylamido-2-methylpropane sulfonic acid (AMPS), ethyl acrylate, ethylene-methyl acrylate, methyl methacrylate, 2-chlorotheyl vinyl ether. 2-hydroxethyl acrylate, hydroxyethyl methacrylate, butyl acrylate, trimethylolpropane triacrylate (TMPTA), amides, such as acrylamide or acrylic acid, dicarboxylic acids, such as itaconic acid or methylidene succinic acid and combinations thereof. In an exemplary embodiment, the monomer comprises AMPS.

The filler in hydrogel layer 40 may comprise any suitable filler material, such as glycerol, hyaluronic acid, and the like.

In one embodiment, the hydrogel layer 40 comprises about 30% to about 60% monomer, such as AMPS, by weight, about 10% to about 50% water by weight and about 10% to about 40% filler, such as glycerol, by weight. In an exemplary embodiment, hydrogel layer 40 comprises 40% AMPS, 30% water and 30% glycerol by weight.

In certain embodiments, hydrogel layer may further include an acrylic acid to increase the tackiness of the gel. In one such embodiment, hydrogel layer 40 includes an acrylic acid in about 1% to about 10%, or about 5% by weight of the layer 40.

In certain embodiments, hydrogel layer 40 may further include one or more materials that facilitate conductivity, such as potassium salt of 3-sulfopropyl ester acrylate (SPA) in about 10% by weight.

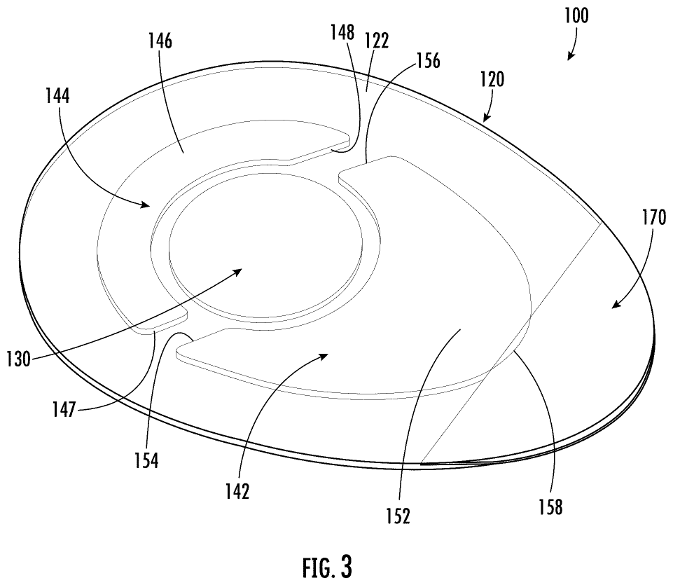

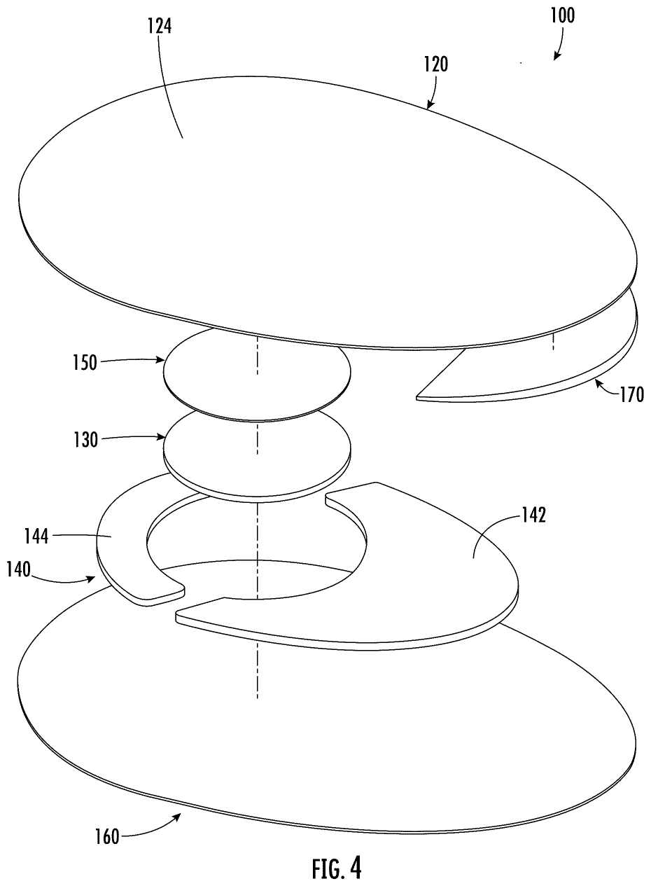

Referring now to FIGS. 3 and 4, another embodiment of a dressing or medicated pad 100 will now be described. As shown, pad 100 includes a first layer 120 having an outer surface 124 and an adhesive surface 122 configured to adhere to a user's skin. Pad 100 further includes a release layer 160 configured to removably attach to adhesive surface 122 and a release tab 170 to allow the user to remove release liner 170 before applying adhesive surface 122 to the skin.

Pad 100 is particularly useful for removing a callus. First layer 120 and release layer 160 generally have an ovoid shape, although it will be recognized that other shapes are contemplated. The ovoid shape allows for more flexibility and adds cushioning when the dressing is placed in certain areas of the foot. In addition, the ovoid shape better conforms to common application areas for calluses.

Medicated pad 100 further includes a second layer 130 that includes an active agent, such as salicylic acid, suitable for removing a target region of a user's skin, such as a corn, callus, blister or the like. Medicated pad 100 includes an interlayer 150 disposed between second layer 130 and outer layer 120. Interlayer 150 functions to protect outer layer 120 from the active agent within second layer 130 such that the active agent does not degrade outer layer 120. Interlayer 150 preferably has the same shape as second layer 130, although it will be recognized that interlayer 150 may include any suitable shape so long as it substantially inhibits or prevents contact between second layer 130 and outer layer 120.

Medicated layer 100 further includes a hydrogel layer 140 disposed between outer layer 120 and release liner 160. Hydrogel layer 140 provides moisture to the user's skin in the area immediately surrounding the target region. Hydrogel layer 140 at least partially surrounds second layer 130 and is spaced from second layer 130 to reduce the amount of pressure applied to the target region of the user's skin.

In one embodiment, hydrogel layer 140 comprises first and second components 142, 144 positioned on opposite sides of second layer 130. First component 142 comprises a generally arcuate main body with a thicker central portion 158 than the end portions 154, 156 to substantially correspond with the ovoid shape of first layer 120. Second component 144 generally has a similar construction as first component 42 of the previous embodiment, comprising a generally arcuate main body 146 with first and second ends 147, 148. The ends 147, 18 of first component 142 are preferably spaced from the ends 154, 156 of second component 144 to form two separately spaced components that substantially surround second layer 130.

Other configurations of hydrogel layer 140 are contemplated. For example, hydrogel layer 140 may comprise a single component that extends around the entire periphery of second layer 130. This single component may comprise one or more thicker portions such that it substantially corresponds to the overall ovoid shape of medicated pad 100. Alternatively, the single component may extend around only a portion of the second layer 130, i.e., from about 180 degrees to about 360 degrees. In other embodiments, hydrogel layer 140 may comprise more than two components. For example, hydrogel layer may comprise a plurality of smaller hydrogel components circumferentially spaced from each other around medicated layer 130.

### EXAMPLES

Applicant conducted stability testing for a number of different medical dressings: (1) un-pouched corn remover dressings with a similar construction as the dressing described above in reference to FIGS. 1 and 2 and having a hydrogel layer containing 50% water content by weight (labeled un-pouched OSD Corn); (2) pouched corn remover dressings with a similar construction as the dressing described above in reference to FIGS. 1 and 2 and having a hydrogel layer containing 50% water content by weight (labeled pouched OSD Corn); (3) a prior art Duragel corn remover as a control; (4) un-pouched callus remover dressings with a similar construction as the dressing described above in reference to FIGS. 3 and 4 having a hydrogel layer containing 30% water content by weight (labeled un-pouched OSD Callus); (5) pouched callus remover dressings with a similar construction as the dressing described above in reference to FIGS. 3 and 4 having a hydrogel layer containing 30% water content by weight (labeled un-pouched OSD Callus); and (6) a prior art Duragel callus remover as a control.

The prior art Duragel corn and callus removers that were used as controls included two separate components: (1) a medical dressing containing a hydrogel on the underlying adhesive surface; and (2) a separate medicated disc containing salicylic acid that is designed to be applied to the user's skin prior to application of the medical dressing. The hydrogel on the medical dressing in the control samples is disposed between the dressing and the medicated disc.

All samples were stored in a 40° C./75% RH stability chamber and inspected to determine if the concentration of salicylic acid and the integrity of the products was compromised by exposure to heat and humidity. The testing occurred over a series of time frames: (1) initial; (2) 1 month; (3) 2 months; (4) 3 months; and (5) 6 months. The samples were visually tested for attribute acceptance criteria that included: (1) whether the hydrogel appeared dried out or crusty; (2) whether there was any discoloration of the hydrogel from the salicylic acid disc; and (3) whether each bandage was easily removed and there was no evidence of residual product or product interaction left on the liner. The samples were also tested analytically to determine whether the salicylic acid in the disc met the acceptance criteria of 38% to 42% by weight of the disc.

Both configurations of the OSC Callus product (pouched and un-pouched) passed all visual attribute testing at all time frames up to six months. In addition, the concentration of salicylic acid remained within the acceptance criteria of 38% to 42% by weight after six months. Both configurations of the OSC Corn product (pouched and un-pouched) failed the visual attribute and the analytical testing prior to the six month period. The control samples passed all visual attribute and analytical testing at all time frames up to six months.

Applicant analyzed the failures of the OSC Corn product and determined that the failures were primarily (if not entirely) caused by the higher concentration (50%) of water in the hydrogel. This higher concentration of water caused degradation of the salicylic acid in the medicated layer to levels below the acceptance criteria of 38%. In addition, this higher concentration of water caused the products to fail various visual attributing tests during this period. Thus, this testing demonstrates that a hydrogel layer containing a water content of less than 50%, in particular 30%, by weight provides distinct advantages to the stability of the dressing.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in a first aspect, a first embodiment is a dressing comprising a first layer having an adhesive surface, a second layer comprising an active agent, the second layer being disposed adjacent to the adhesive surface of the first layer and a third layer comprising hydrogel spaced from and at least partially surrounding the second layer.

A second embodiment is the first embodiment, wherein the second layer is spaced from the first layer and the third layer is in contact with the first layer.

A third embodiment is any combination of the first two embodiments, wherein the active agent comprises salicylic acid.

A 4^{th} embodiment is any combination of the first 3 embodiments, wherein the hydrogel comprises water in a percentage of less than about 50% by weight of the third layer.

A 5^{th} embodiment is any combination of the first 4 embodiments, wherein the hydrogel comprises water in a percentage of about 10% to about 50% by weight of the third layer

A 6^{th} embodiment is any combination of the first 5 embodiments, wherein the percentage is less than about 40%.

A 7^{th} embodiment is any combination of the first 6 embodiments, wherein the percentage is about 30%.

An 8^{th} embodiment is any combination of the first 7 embodiments, wherein the third layer further comprises an acrylic based monomer.

A 9^{th} embodiment is any combination of the first 8 embodiments, wherein the third layer further comprises a filler.

A 10^{th} embodiment is any combination of the first 9 embodiments, wherein the third layer comprises a first component comprising hydrogel and a second component comprising hydrogel, wherein the first component is spaced from the second component on an opposite side of the second layer.

An 11^{th} embodiment is any combination of the first 10 embodiments, wherein the first component has substantially the same shape as the second component.

A 12^{th} embodiment is any combination of the first 11 embodiments, wherein the first component has a first cross-sectional area and the second component has a second cross-sectional area, wherein the first cross-sectional area is larger than the second cross-sectional area.

A 13^{th} embodiment is any combination of the first 12 embodiments, wherein the outer layer comprises a material selected from a group consisting of polyurethane, polyolefin, polyamide, polyester, polypropylene, polyethylene, ethylene vinyl acetate (EVA) and combinations thereof.

A 14^{th} embodiment is any combination of the first 13 embodiments, further comprising an interlayer between the adhesive surface of the first layer and the second layer.

A 15^{th} embodiment is any combination of the first 14 embodiments, wherein the interlayer comprises a material selected from a group consisting of polyethylene terephthalate (PET), polyurethane, polyolefin, polyamide, polyester, polypropylene, polyethylene, ethylene vinyl acetate (EVA) and combinations thereof.

A 16^{th} embodiment is any combination of the first 15 embodiments, further comprising a fourth layer removably coupled to the first layer, wherein the second layer is disposed between the first layer and the fourth layer.

A 17^{th} embodiment is any combination of the first 16 embodiments, wherein the second layer contacts the fourth layer.

An 18^{th} embodiment is any combination of the first 17 embodiments, wherein the third layer contacts the first layer and the fourth layer.

A 19^{th} embodiment is any combination of the first 18 embodiments, wherein the fourth layer comprises a material selected from a group consisting of polyethylene terephthalate (PET), polyurethane, polyolefin, polyamide, polyester, polypropylene, polyethylene, ethylene vinyl acetate (EVA) and combinations thereof.

A 20^{th} embodiment is any combination of the first 19 embodiments, further comprising a release tab coupled to the first layer for removing the fourth layer.

A 21^{st} embodiment is any combination of the first 20 embodiments, wherein the second layer comprises a substantially circular disc.

A 22^{nd} embodiment is any combination of the first 21 embodiments, wherein the third layer comprises a first component and a second component spaced from the first component, wherein the first and second components each comprise a semi-annular shape disposed on opposite sides of the second layer.

A 23^{rd} embodiment is any combination of the first 22 embodiments, wherein the first and second components of the third layer each have an arcuate main body with first and second ends, wherein the first end of the first component is spaced from the first end of the second component and the second end of the first component is spaced from the second end of the second component.

In another aspect, a medical device is provided with the dressing of any of the first 23 embodiments.

In another aspect, a corn remover is provided with the dressing of any of the first 23 embodiments.

In another aspect, a callus remover is provided with the dressing of any of the first 23 embodiments.

In another aspect, a blister remover is provided with the dressing of any of the first 23 embodiments.

In another aspect, a wart remover is provided with the dressing of any of the first 23 embodiments.

In another aspect, a first embodiment is a medicated pad for use in removing a corn from a user's skin. The medicated pad comprises a first layer having an adhesive surface configured for contacting the user's skin, a second layer comprising an active agent configured to remove the corn and a third layer comprising hydrogel and spaced from and at least partially surrounding the second layer.

A second embodiment is the first embodiment, wherein the second layer comprises a substantially circular disc.

A 3^{rd} embodiment is any combination of the first 2 embodiments, wherein the third layer comprises a first component and a second component spaced from the first component, wherein the first and second components each comprise a semi-annular shape disposed on opposite sides of the circular disc.

A 4^{th} embodiment is any combination of the first 3 embodiments, wherein the active agent comprises salicylic acid.

A 5^{th} embodiment is any combination of the first 4 embodiments, wherein the hydrogel comprises water in a percentage of less than about 50% by weight of the second layer.

A 6^{th} embodiment is any combination of the first 5 embodiments, wherein the hydrogel comprises water in a percentage of about 10% to about 50% by weight of the third layer

A 7^{th} embodiment is any combination of the first 6 embodiments, wherein the percentage is less than about 40%.

An 8^{th} embodiment is any combination of the first 7 embodiments, wherein the percentage is about 30%.

A 9^{th} embodiment is any combination of the first 8 embodiments, wherein the hydrogel further comprises an acrylic based monomer.

A 10^{th} embodiment is any combination of the first 9 embodiments, further comprising an interlayer between the adhesive surface of the first layer and the second layer.

An 11^{th} embodiment is any combination of the first 10 embodiments, further comprising a fourth layer, wherein the second layer is disposed between the first layer and the fourth layer.

A 12^{th} embodiment is any combination of the first 11 embodiments, wherein the second layer contacts the fourth layer.

A 13^{th} embodiment is any combination of the first 12 embodiments, wherein the third layer contacts the first layer and the fourth layer.

In another aspect, a first embodiment is a medicated pad for use in removing a callus from a user's skin. The medicated pad comprises a first layer having an adhesive surface configured for contacting the user's skin, a second layer comprising an active agent configured to remove the callus and a third layer comprising hydrogel and spaced from and at least partially surrounding the second layer.

A second embodiment is the first embodiment, wherein the outer layer comprises a substantially ovoid shape.

A 3^{rd} embodiment is any combination of the first 2 embodiments, wherein the second layer comprises a substantially circular disc.

A 4^{th} embodiment is any combination of the first 3 embodiments, wherein the third layer comprises a first component and a second component spaced from the first component, wherein the first and second components are disposed on either side of the circular disc and the first component has a smaller cross-sectional area than the second component.

A 5^{th} embodiment is any combination of the first 4 embodiments, wherein the second component comprises first and second end portions and a central portion, wherein the central portion has a larger thickness than the first and second end portions.

A 6^{th} embodiment is any combination of the first 5 embodiments, wherein the active agent comprises salicylic acid.

A 7^{th} embodiment is any combination of the first 6 embodiments, wherein the hydrogel comprises water in a percentage of less than about 50% by weight of the second layer.

An 8^{th} embodiment is any combination of the first 7 embodiments, wherein the hydrogel comprises water in a percentage of about 10% to about 50% by weight of the third layer.

A 9^{th} embodiment is any combination of the first 8 embodiments, wherein the percentage is less than about 40%.

A 10^{th} embodiment is any combination of the first 9 embodiments, wherein the percentage is about 30%.

An 11^{th} embodiment is any combination of the first 10 embodiments, wherein the hydrogel further comprises an acrylic based monomer.

A 12^{th} embodiment is any combination of the first 11 embodiments, further comprising an interlayer between the adhesive surface of the first layer and the second layer.

A 13^{th} embodiment is any combination of the first 12 embodiments, further comprising a fourth layer, wherein the second layer is disposed between the first layer and the fourth layer.

A 14^{th} embodiment is any combination of the first 13 embodiments, wherein the second layer contacts the fourth layer.

A 15^{th} embodiment is any combination of the first 14 embodiments, wherein the third layer contacts the first layer and the fourth layer.

1. A dressing comprising:
a first layer having an adhesive surface;
a second layer comprising an active agent, the second layer being disposed adjacent to the adhesive surface of the first layer; and
a third layer comprising hydrogel spaced from and at least partially surrounding the second layer.

2. The dressing of claim 1, wherein the second layer is spaced from the first layer and the third layer is in contact with the first layer.

3. The dressing of claim 1, wherein the active agent comprises salicylic acid.

4. The dressing of claim 1, wherein the hydrogel comprises water in a percentage of about 10% to about 50% by weight of the third layer.

5. The dressing of claim 4, wherein the percentage is less than about 40%.

6. The dressing of claim 4, wherein the percentage is about 30%.

7. The dressing of claim 1, wherein the third layer further comprises an acrylic based monomer.

8. The dressing of claim 1, wherein the third layer further comprises a filler.

9. The dressing of claim 1, wherein the third layer comprises a first component comprising hydrogel and a second component comprising hydrogel, wherein the first component is spaced from the second component on an opposite side of the second layer.

10. The dressing of claim 9, wherein the first component has substantially the same shape as the second component.

11. The dressing of claim 9, wherein the first component has a first cross-sectional area and the second component has a second cross-sectional area, wherein the first cross-sectional area is larger than the second cross-sectional area.

12. The dressing of claim 1, wherein the outer layer comprises a material selected from a group consisting of polyurethane, polyolefin, polyamide, polyester, polypropylene, polyethylene, ethylene vinyl acetate (EVA) and combinations thereof.

13. The dressing of claim 1, further comprising an interlayer between the adhesive surface of the first layer and the second layer, wherein the interlayer comprises a material selected from a group consisting of polyethylene terephthalate (PET), polyurethane, polyolefin, polyamide, polyester, polypropylene, polyethylene, ethylene vinyl acetate (EVA) and combinations thereof.

14. The dressing of claim 1, further comprising a fourth layer removably coupled to the first layer, wherein the second layer is disposed between the first layer and the fourth layer, wherein the second layer contacts the fourth layer and the third layer contacts the first layer and the fourth layer.

15. The dressing of claim 14, wherein the fourth layer comprises a material selected from a group consisting of polyethylene terephthalate (PET), polyurethane, polyolefin, polyamide, polyester, polypropylene, polyethylene, ethylene vinyl acetate (EVA) and combinations thereof.

16. The dressing of claim 1, further comprising a release tab coupled to the first layer for removing the fourth layer.

17. The dressing of claim 1, wherein the second layer comprises a substantially circular disc.

18. The dressing of claim 17, wherein the third layer comprises a first component and a second component spaced from the first component, wherein the first and second components each comprise a semi-annular shape disposed on opposite sides of the second layer.

19. The dressing of claim 18, wherein the first and second components of the third layer each have an arcuate main body with first and second ends, wherein the first end of the first component is spaced from the first end of the second component and the second end of the first component is spaced from the second end of the second component.

20. A corn remover comprising the dressing of claim 1.

21. A callus remover comprising the dressing of claim 1.
